(19) United States
(12) Patent Application Publication
(10) Pub. No.: US 2022/0387690 A1
(43) Pub. Date: Dec. 8, 2022

(54) ARTERIOVENOUS GRAFT WITH EXCHANGEABLE INNER LUMEN

(71) Applicant: Ohio State Innovation Foundation, Columbus, OH (US)

(72) Inventors: Mark Ruegsegger, Columbus, OH (US); Khaled Boubes, Columbus, OH (US); Sheila Colbert, Columbus, OH (US); Gurleen Vilkhu, Columbus, OH (US); Jacob Miller, Columbus, OH (US); Kellen Biesbrock, Columbus, OH (US); Ana Minyayev, Columbus, OH (US); Kenzington Kottenbrock, Columbus, OH (US); Jordan Rosales, Columbus, OH (US); Tasneem Mohammad, Columbus, OH (US); Keerthi Kumar, Columbus, OH (US)

(21) Appl. No.: 17/728,650

(22) Filed: Apr. 25, 2022

Related U.S. Application Data
(60) Provisional application No. 63/178,916, filed on Apr. 23, 2021.

(51) Int. Cl. A61M 1/36; A61M 25/00

(52) U.S. Cl. CPC A61M 1/3655; A61M 1/3661; A61M 25/0026; A61M 2205/0266; A61M 2205/0272; A61M 25/104

(57) ABSTRACT

Various implementations include an arteriovenous graft device. The device includes an outer conduit and an inner conduit. The outer conduit has a first outer conduit end and a second outer conduit end opposite the first outer conduit end. The first outer conduit end defines an outer conduit opening extending from the first outer conduit end to the second outer conduit end. The first outer conduit end is configured to be in fluid communication with an artery and the second outer conduit end is configured to be in fluid communication with a vein. The inner conduit defines an inner conduit opening. The inner conduit is sized to be disposed within the outer conduit opening. The inner conduit is movable between a collapsed position and an expandable position.

### CROSS-REFERENCE TO RELATED APPLICATIONS

This application claims the benefit of U.S. Provisional Patent Application No. 63/178,916, filed Apr. 23, 2021, the content of which is incorporated herein by reference in its entirety.

### BACKGROUND

Kidney disease is any condition that causes a reduction in kidney function. There are five stages of kidney disease and in end stage kidney disease, patients require dialysis treatment to filter waste and water from the blood as their kidneys can no longer function without assistance. Chronic kidney disease is a gradual decline in kidney function over time that can eventually progress to end-stage renal disease (ESRD) which is total kidney failure. The majority of patients with kidney failure receive hemodialysis for treatment in which blood flows from an arterial needle to a dialyzer where it is filtered; filtered blood returns to the body through a venous needle.

The hemodialysis dialyzer requires vascular access, typically via a surgically-created vein, for the removal and return of blood. For hemodialysis treatment, two needles, which are attached to soft tubes connected to the dialyzer, are placed into the patient's arm using the vascular access. Blood flows from the arterial needle from the vascular access to the dialyzer, where it is filtered. This filtered blood then returns to the patient's body via the venous needle

There are three common ways of accessing the patient's vasculature for dialysis: the central-venous catheter (CVC), the arteriovenous fistula (AVF), and the arteriovenous graft (AVG). Diagrams of these three access methods with the arterial and venous connections are shown in FIGS. 1A-1C.

FIG. 1A illustrates tunneled venous catheter, in which the CVC is a flexible tube placed through the skin into the patient's neck, chest, or groin, allowing for immediate access for hemodialysis. FIG. 1B illustrates AVF in a patient's forearm, in which a surgical connection between the patient's vein and artery that enlarges the vein, allowing the needle to be placed sufficiently for dialysis. The AVF requires a lengthy maturation period before it is strong enough to be used for dialysis, but it is the preferred method of vascular access because it lasts longer and results in fewer complications when compared with the other methods. However, when the patient lacks a suitable vein for creation of a fistula, the AVG is used for vascular access for dialysis.

FIG. 1C illustrates an arteriovenous graft (AVG) in a patient's forearm. To form an AVG, a surgeon uses a subdermal, synthetic tube to connect the patient's artery and vein, and feed the high volume of blood within the artery through the smaller vein. Two needles can then be inserted into the graft material for hemodialysis, as described above. Of the patients receiving hemodialysis treatment, about 15% utilize an arteriovenous graft (AVG) as the mode of vascular access. Complications of the AVG include stenosis, thrombosis, and pseudoaneurysms, which impede blood flow needed for hemodialysis and require surgical intervention frequently, e.g., within three to four months of initial graft placement.

Stenosis is the narrowing of any blood vessel and can occur in the AVG as endothelial cells buildup along the inner layer. Thrombosis is a blood clot that occurs within the AVG and causes obstruction of blood flow. A pseudoaneurysm occurs when the blood is able to escape from the graft, typically from the puncture sites, and begins to collect in surrounding tissue. These complications require surgical intervention approximately every three to four months after the graft's first year in place.

Referring to FIG. 2, balloon angioplasty is the desired method for AVG intervention, specifically for stenosis complications. The balloon angioplasty process includes the insertion of a catheter 202 with a balloon 204 attached to its end. When the balloon 202 reaches the area of stenosis 208, it is inflated in order to widen the vasculature to the desired amount. In FIG. 2, balloon angioplasty is displayed in a narrowed blood vessel 206 in the same way this is done to open a graft. Surgeons prefer not to use stent placement in treating AVG complications in order to preserve the native veins for possible future fistula creation. The repetitive puncture the AVG experiences for dialysis approximately three times a week is also a stimulant for complication as the puncturing induces endothelial cell buildup within the graft.

Accordingly, there is a need for a device that minimizes the complications of an arteriovenous graft and allows an arteriovenous graft to be used for longer without requiring surgical intervention.

### BRIEF SUMMARY OF THE DISCLOSURE

Accordingly, the present invention is directed to a vascular graft with an interchangeable inner lumen that obviates one or more of the problems due to limitations and disadvantages of the related art.

In accordance with the purpose(s) of this invention, as embodied and broadly described herein, this invention, in one aspect, relates to an arteriovenous graft device having an outer conduit having a first outer conduit end and a second outer conduit end opposite the first outer conduit end, the first outer conduit end defining an outer conduit opening extending from the first outer conduit end to the second outer conduit end, wherein the first outer conduit end is configured to be in fluid communication with an artery and the second outer conduit end is configured to be in fluid communication with a vein; and an inner conduit defining an inner conduit opening, wherein the inner conduit is sized to be disposed within the outer conduit opening, wherein the inner conduit is movable between a collapsed position and an expandable position.

In another aspect, the invention relates to a method of inserting an inner conduit into an outer conduit of an arteriovenous graft device including providing an outer conduit having a first outer conduit end and a second outer conduit end opposite the first outer conduit end, the first outer conduit end defining an outer conduit opening extending from the first outer conduit end to the second outer conduit end, wherein the first outer conduit end is in fluid communication with an artery and the second outer conduit end is in fluid communication with a vein; advancing an inner conduit coupled to a guidewire through a portion of the artery to the first outer conduit end, the inner conduit defining an inner conduit opening, wherein the inner conduit is sized to be disposed within the outer conduit opening, wherein the inner conduit is in a collapsed position and is movable between a collapsed position and an expandable position; inserting the inner conduit into the outer conduit opening; and moving the inner conduit from the collapsed position to the expanded position.

In yet another aspect, the invention relates to a method of removing an inner conduit from an outer conduit of an arteriovenous graft device including providing an arteriovenous graft device, the device having an outer conduit having a first outer conduit end and a second outer conduit end opposite the first outer conduit end, the first outer conduit end defining an outer conduit opening extending from the first outer conduit end to the second outer conduit end, wherein the first outer conduit end is in fluid communication with an artery and the second outer conduit end is in fluid communication with a vein, and an inner conduit defining an inner conduit opening, wherein the inner conduit is disposed within the outer conduit opening, wherein the inner conduit is in an expanded position and is movable between a collapsed position and an expandable position; advancing a guidewire through a portion of the artery to the inner conduit; coupling the guidewire to the inner conduit; moving the inner conduit from the expanded position to the collapsed position; removing the inner conduit from the outer conduit opening; and removing the guidewire and inner conduit from the artery

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

Further embodiments, features, and advantages of the vascular graft with an interchangeable inner lumen, as well as the structure and operation of the various embodiments of the vascular graft with an interchangeable inner lumen are described in detail below with reference to the accompanying drawings.

It is to be understood that both the foregoing general description and the following detailed description are exemplary and explanatory only, and are not restrictive of the invention as claimed.

### BRIEF DESCRIPTION OF THE DRAWINGS

Example features and implementations are disclosed in the accompanying drawings. However, the present disclosure is not limited to the precise arrangements and instrumentalities shown. The accompanying figures, which are incorporated herein and form part of the specification, illustrate the vascular graft with an interchangeable inner lumen. Together with the description, the figures further serve to explain the principles of the vascular graft with an interchangeable inner lumen described herein and thereby enable a person skilled in the pertinent art to make and use the vascular graft with an interchangeable inner lumen.

FIGS. 1A-1C illustrates various access methods for arterial and venous connections for hemodialysis.

FIG. 2 illustrates balloon angioplasty for intervention of complication caused by arteriovenous grafts for hemodialysis.

FIG. 3 illustrates an embodiment of a vascular graft with an interchangeable inner lumen according to principles described herein.

FIGS. 4 and 5 illustrates another embodiment of a vascular graft with an interchangeable inner lumen according to principles described herein

FIG. 6 illustrates a method of removal of an inner lumen in a vascular graft with an interchangeable inner lumen according to principles described herein.

FIG. 7 illustrates another embodiment of a vascular graft with an interchangeable inner lumen according to principles described herein.

FIG. 8 illustrates another embodiment of a vascular graft with an interchangeable inner lumen according to principles described herein

FIG. 9 illustrates another embodiment of a vascular graft with an interchangeable inner lumen according to principles described herein.

FIG. 10 illustrates another embodiment of a vascular graft with an interchangeable inner lumen according to principles described herein.

FIG. 11 illustrates another embodiment of a vascular graft with an interchangeable inner lumen according to principles described herein.

FIG. 12 illustrates another embodiment of a vascular graft with an interchangeable inner lumen according to principles described herein.

FIG. 13 illustrates a test set up for embodiments of a vascular graft according to principles described herein.

### DETAILED DESCRIPTION

Reference will now be made in detail to embodiments of the vascular graft with an interchangeable inner lumen with reference to the accompanying figures. The same reference numbers in different drawings may identify the same or similar elements.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

Disclosed herein is a vascular graft with an interchangeable lumen that can be inserted and replaced endovascularly, reducing the frequency and invasiveness of surgical intervention that is needed for current arteriovenous grafts (AVGs). Disclosed herein is also a method of allowing various implementations of the disclosed device to be removed and replaced by a new implementation of the disclosed device when wear and obstruction issues are faced, thereby decreasing the frequency of intervention.

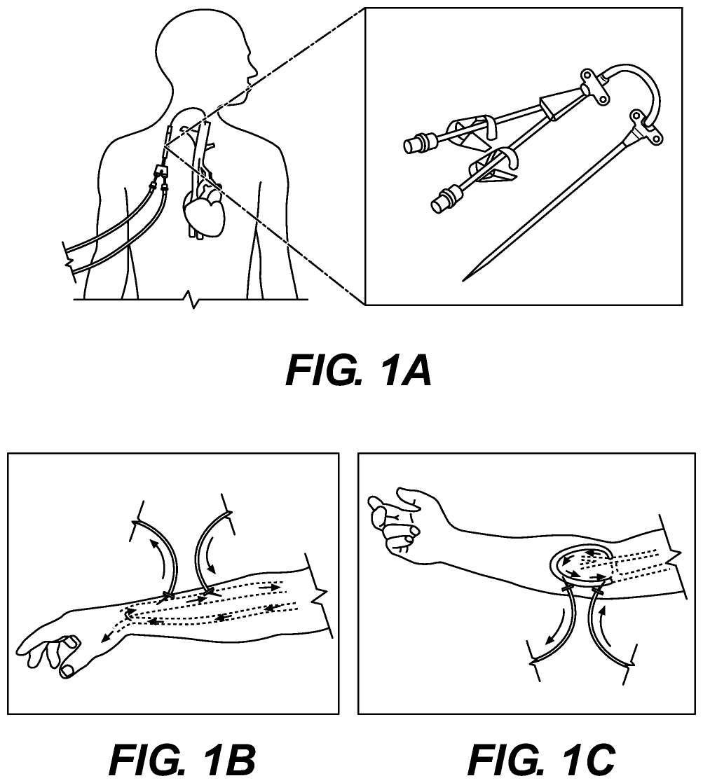

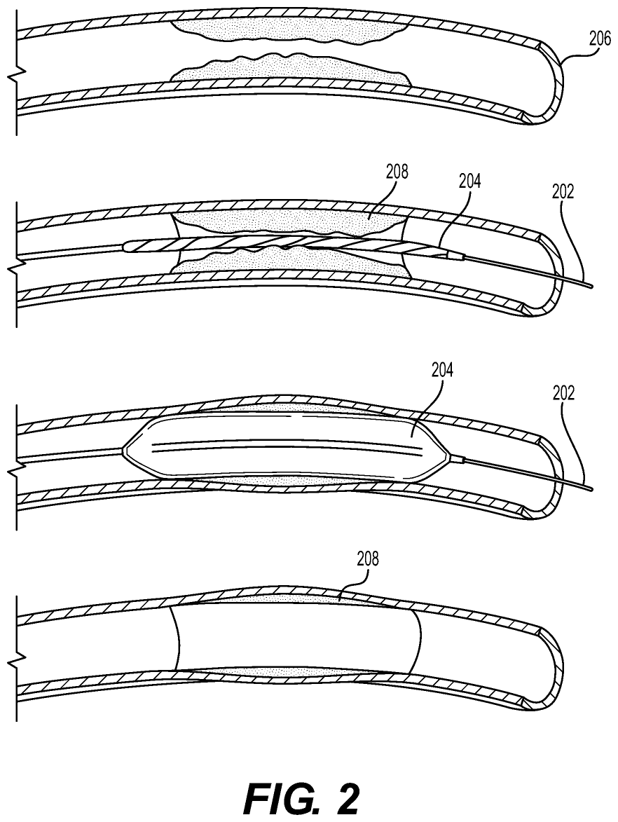

In an AVG, a synthetic conduit is used to connect the artery and vein instead of natural vein. An AVG is typically placed in the arm of the patient in a straight or looped manner depending on the anatomy of the patient. (See FIG. 1C). A straight positioned graft is preferred for ease of puncturing and because loops can encourage stenosis. The graft is placed using local anesthesia and two small incisions to allow for access to the artery and vein.

The most common materials used in the AVG include polytetrafluoroethylene (PTFE) and polyurethane, although there are also some applications of bovine carotid artery. PTFE is a polymer comprising carbon and fluorine and is highly hydrophobic. Polyurethane is a polymer comprising carbamate links, can be thermosetting or thermoplastic, and can be manufactured to be either hydrophobic or hydrophilic. In a standard AVG made from PTFE, the graft is approximately 70 cm in length and then is cut by the surgeon to the required length depending on the needs and available vasculature of the specific patient. Unlike the two to three-month AVF maturation period, the AVG has only a two-week maturation period where the graft material becomes incorporated into the tissue before it can be used

The disclosed device includes an inner lumen for the AVG that is configured to be placed and removed endovascularly to decrease the cell build up and complications of the AVG and therefore, decrease the amount of surgical intervention required. While the disclosure utilizes the example of hemodialysis, in other implementations, the disclosed device is used for any other type of dialysis treatment or within any other types of grafts. In other implementations, the disclosed device is configured to be inserted into a groin graft.

The disclosed graft-lumen device includes an inner lumen that is collapsible to allow for removal endovascularly that also maintains its shape so it does not interfere with blood flow through the graft. In addition, the graft-lumen device is able to withstand repetitive puncturing and moreover, seal the holes left behind from puncturing to prevent bleeding. The graft-lumen device also includes biocompatible material such that the graft-lumen device is biocompatible with endothelial cells and non-biodegradable. Lastly, the graft-lumen device is able to withstand shear forces from blood flow, extend for a length long enough to connect an artery and vein, and have a lifespan of at least 6 months or more.

The graft-lumen device disclosed herein is replaceable, collapsible inside a graft shell, able to retain its respective shape and structure in vivo, has a lifespan of at least six months, and is inserted and removed endovascularly. In some implementations, the graft-lumen device also includes biocompatible materials.

In some embodiment, the graft-lumen device may be of any diameter to provide a good blood circulation flow and hemodialysis flow rate through the dialysis machine. For example, a 1000 mL/min through the graft and a 400 mL/min hemodialysis flow rate through the dialysis machine may be achieved with a graft-lumen device having a standard inner radius of 6-8 mm.

Also, to raise patient satisfaction, the life span of the graft's lumen must be longer than the time interval between interventions to clear/clean the grafts, the graft-lumen device should withstand repetitive puncturing by two needles approximately two to three times a week, and reduce the risk of stenosis, thrombosis, and pseudoaneurysms due to puncturing.

In various aspects, the graft lumen may maintain its radial tension and adhere to the inner wall of the AV graft to prevent the migration of endothelial cells between the graft and the lumen, maintain its axial tension preventing the formation of crimps at curvatures and/or under the shear force of blood flow, and have the same length as the AV graft. In another aspect, the graft-lumen device may maintain its shape for the desired lifespan.

In some implementations, the graft-lumen device includes a net-like structure incorporated into a membrane which can vary the inner topography of the lumen. In some implementations, the life span of the graft lumen device is greater than eight or greater than twelve months. In some implementations, the replacement of the graft-lumen device is easier and includes using an electromagnet that collapses and removes/places the graft-lumen device while the AV graft blocks the blood flow at venous end.

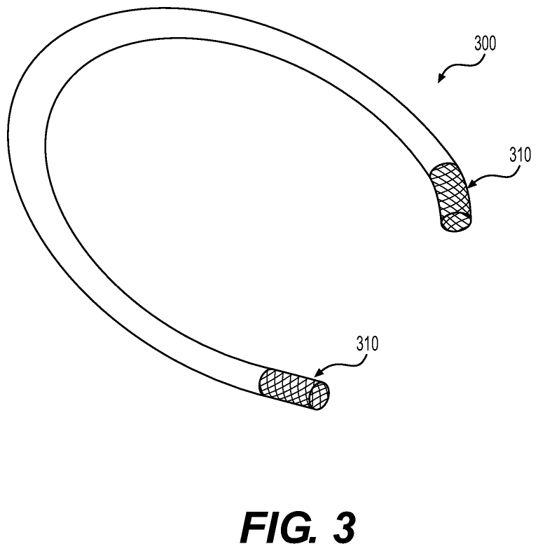

In one implementation, as shown in FIG. 3, the graft-lumen device includes a traditional PTFE graft 307, cylindrical and thin, with a nitinol stent 310 on either end. The nitinol stents on either end of the device are temperature dependent and allow for removal by running iced-saline solution through the graft lumen. Due to the shape-memory property of nitinol, by cooling the metal, it will return to a pre-made, thinner geometry. The result will have the stent peel away from the graft shell, as well as cause the contraction of the PTFE component attached to the stent. The narrowing will allow the endovascular removal of the now thinned inner lumen by use of a guide wire (not shown).

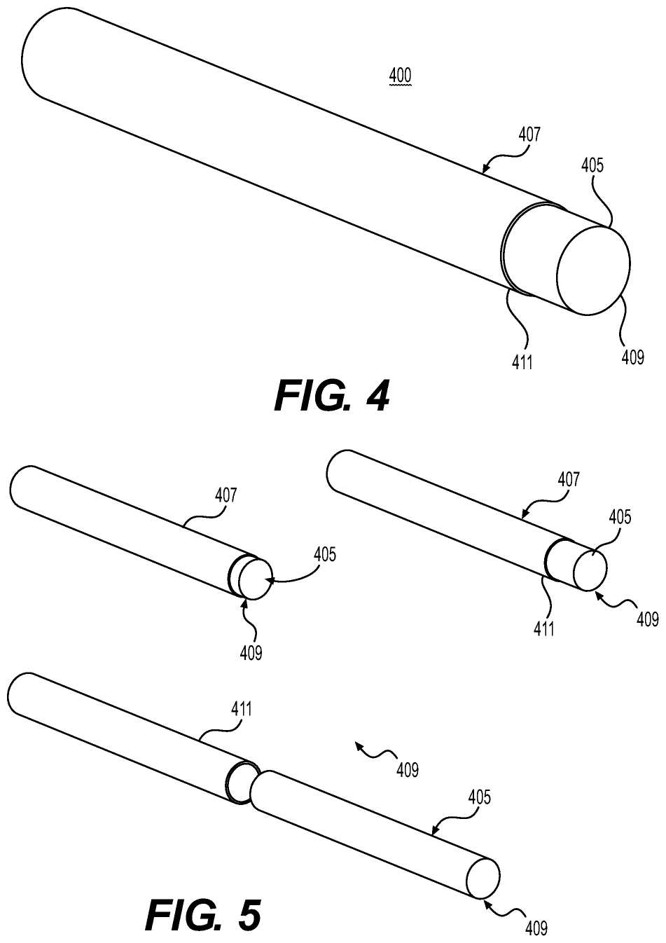

FIG. 4 illustrates another implementation of the graft-lumen device 400 including a traditional PTFE graft that is integrated with magnetic nanoparticles. These nanoparticles are not inherently magnetic but can be manipulated under a magnetic field. By coating these nanoparticles in a biocompatible polymer, they can then be integrated into a medical device safely. The geometry of the inner lumen 405 remains a simple cylinder. Once placed inside the outer shell 407, the inner lumen 405 can be manipulated to contract by inserting an electromagnet endovascularly inside the center of the inner lumen 405. Once the electromagnet is activated, the nanoparticles will attract concentrically causing the inner lumen 405 to contract (thin), causing the inner lumen 405 to separate from the outer shell 407 and be thin enough to then be removed endovascularly.

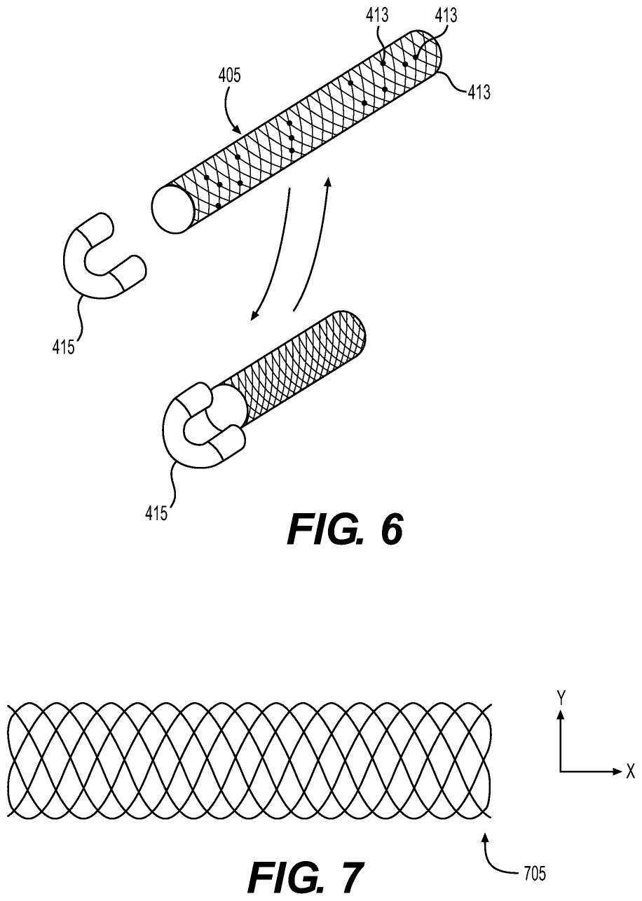

FIG. 4 illustrates magnetic nano particle coated inner lumen 405 (black) in an expanded state and concentric to the PTFE outer lumen 407 (gray). The inner lumen edge 409 sits passed the edge 411 of the outer shell 407 in this figure to facilitate differentiation between layers. In vivo, the shell edge 409 will line up with the edge 411 of the inner lumen 405. Other views and a schematic of the removal of the implementation of the graft-lumen device of FIG. 4 are shown in FIGS. 5 and 6.

In terms of form, the inner lumen 405 of the graft-lumen device shown in FIG. 4 appears as a polymer graft with magnetic particles interwoven in between the strands of PTFE via electrospinning. In an aspect, the device can be placed within the more permanent outer AVG. In terms of function, when complications arise, which currently require full graft replacement, the vascular surgeon can endovascularly remove the graft-lumen device. This reduces the frequency of the more invasive full graft replacement procedure and extends the lifetime of the outer AVG.

The mechanism for removal involves activating the embedded magnetic nanoparticles 413 to shrink the inner lumen's diameter so it can be removed endovascularly. Similarly, when placing a new inner lumen within the existing AVG, the diameter shrinks until it reaches the graft 407, at which point the embedded magnetic nanoparticles 413 may be used to induce a diameter increase to adhere to the outer graft 407.

A magnetic catheter method may involve placing the catheter within the inner lumen 405 endovascularly to induce magnetism while placing or removing the lumen. The external magnet method involves applying a magnet 415 outside of the body and following the lumen 405 as it is placed or removed endovascularly with a guidewire (not shown) to make sure the diameter is sufficiently small for movement through the blood vessels.

The magnetic nanoparticles 413 are a reliable source for the collapsibility of the lumen 405 because the magnetic nanoparticles 413 can induce the change of diameter with acceptable consistency, which allow for reliable endovascular removal. In addition, this implementation allows for puncturability because when the graft-lumen device is not being replaced, the magnetic nanoparticles 413 will not affect the puncturability of the device because they will not be activated and will be spaced apart. Furthermore, this graft-lumen device adheres to the outer layer 407 because the hydrophobicity of the material of the graft-lumen device will induce hydrophobic attractions between the inner and outer layers 405/407 of the AVG, causing adherence of the two layers.

PTFE was chosen as a material for the inner lumen because it is hydrophobic, biocompatible, and elastic. Others suitable biocompatible materials may also be used to provide adherence of the inner lumen to the outer graft to minimize slips and crimps of the lumen 405 that could obstruct blood flow, to promote endothelization to cover up punctures from dialysis treatment, to be able to change shape for removal, and to allow for frequent puncture for dialysis treatment. The magnetic nanoparticles 413 may be, for example, Fe3O4 or FePt because they are magnetic and biocompatible. Other suitable magnetic nanoparticles may be used to provide for removal and replacement via an endovascular procedure. In addition, these properties allow for the maintenance of axial and radial tension to provide for the inner lumen 405 to substantially withstand shear force caused by blood flow.

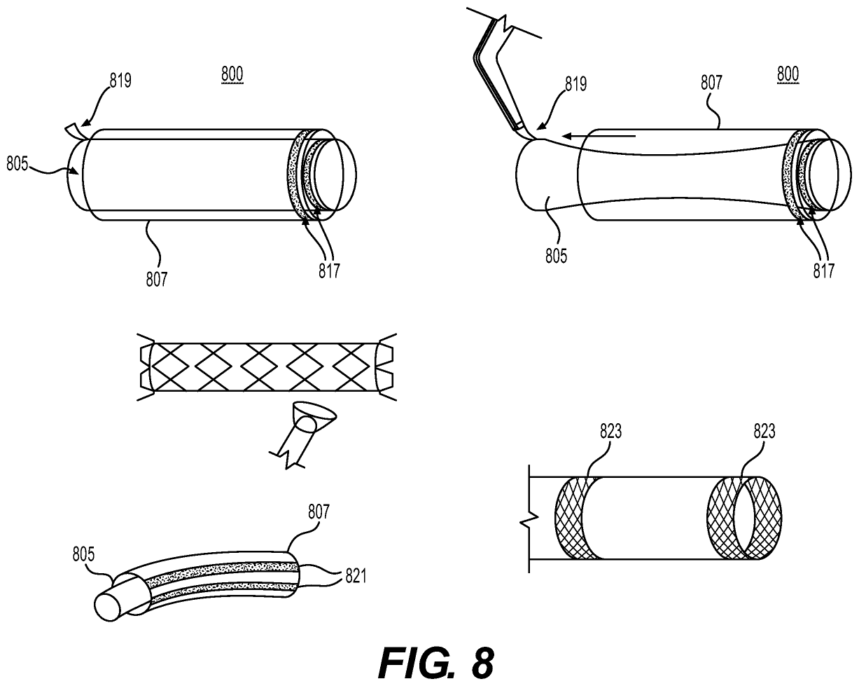

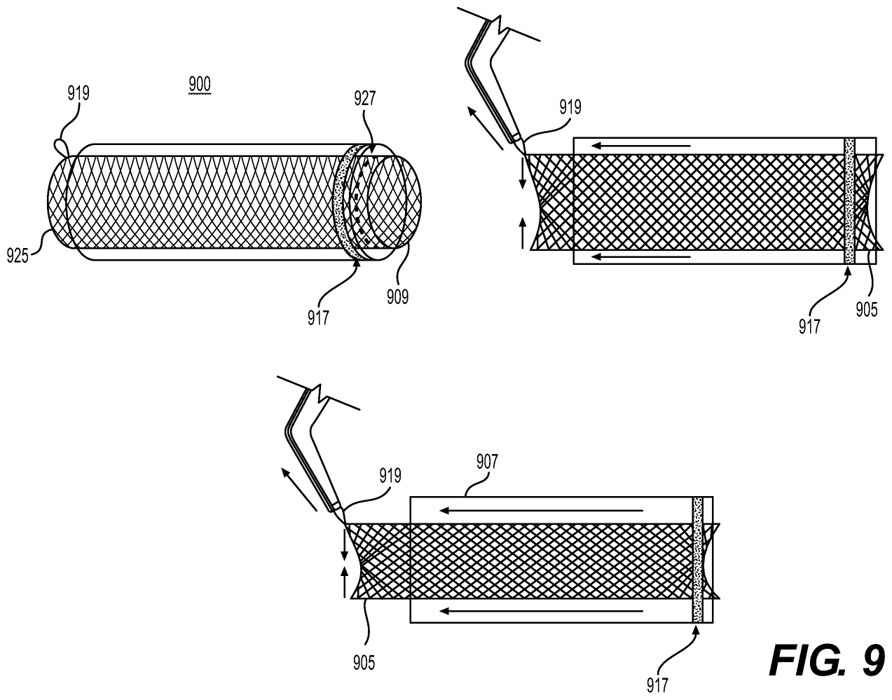

As illustrated in FIG. 7, in yet another implementation, the graft-lumen device may include temperature sensitive polyurethanes that are woven in a method similar to that used in stents to form an inner lumen 705; however, in an aspect, the structure may be more loosely knit than a similar stent, thus creating a less restrictive geometry than the aforementioned implementations. The less restrictive geometry compared to traditional stents allows for a higher freedom of mobility in the lumen, which facilitates the flexible nature needed for in vivo placement of a graft. Additionally, a more loosely knit material allows for the insertion of the larger-tipped needle used during dialysis treatment with a decreased risk in the insertion process damaging the geometry or integrity of the polyurethane structure.

For the removal process of this inner lumen 705, iced saline solution may be run through the graft in vivo, causing the polyurethane polymers to contract (reduce in size). Because of this geometry, a reduction in size for each individual polymer strand translates into the thinning in diameter and reduction in length for the inner lumen 705, allowing for removal of the product by guidewire (not shown). An example of a stented geometry is seen in FIG. 7.

All of the aforementioned implementations are configured with the assumption that each will be coupled to a standard PTFE graft as the outer shell, which allows for easy clinical translation by keeping a familiar component for surgeons wanting to implement the disclosed device. Additionally, all of the aforementioned implementations may use a base material of PTFE for the inner lumen, which allows a hydrophobic interaction between the inner lumen and the outer shell to reduce or to prevent polar materials from getting between the two layers.

In some aspects according to embodiments of the device described herein, the device will have biocompatible properties and not cause adverse reactions, as well as provide a volumetric flow rate for desired dialysis flow rate, e.g., 1000 ml of blood/min in order to support a 400 ml/min flow rate to the dialysis machine. The inner lumen will then maintain its shape and position under the shear force of this blood flow, meaning that it adheres to the inner AVG wall and not wrinkle at curves in the graft. In addition, the device will withstand punctures will allow for endovascular replacement.

The vascular graft according to principles described herein may include additional features, for example, to reduce additional issues that would be caused by the lumen traveling downstream. For example, the surface topography of the device may be smooth in part or throughout the blood/material interface, which may reduce the chance of protein adhesion (which can lead to stenosis, thrombosis, etc) and improve lifespan of the device. In some aspects, the device lifespan may be 6-12 months or more be able to be replaced endovascularly. To aid in the ease of interchanging the inner lumen endovascularly, the device and/or the inner lumen, may contract radially.

Other aspects of a device according to principles described herein are illustrated in FIG. 8. As illustrated in FIG. 8, the device may use neodymium magnetic rings or tabs on one or both ends of the inner lumen 805 and outer graft 807 to ensure the layers adhere well to each other and to act as an anchor against tension during removal. Neodymium magnets feature a high magnetic field density and are often used in dental and orthopedic applications, including as implants for myokinetic prosthetic control. By pulling on a string or removal tab 819 on the opposite end of the inner lumen 805 with forceps or a hook, the tension force initiates narrowing of the inner lumen 805. Increasing the amount of tension frees the magnetic portions of the graft 800 from each other.

Other designs may include different shaped meshes with regularly spaced folds to enable the inner lumen 805 to self-expand during insertion and to aid in uniform collapse during the removal process. Stenting of the inner lumen 805 with stents 823 at known positions or regular intervals may allow for easier collapse and removal, balloon-catheter-based insertion methods, and the use of raised ridges on the outer graft to indicate areas of puncture. The sketch in the bottom left of FIG. 8 shows a magnetic nanoparticle coated inner lumen 805 encased by an outer graft 807 integrated with magnetic ridges 821. The resulting magnetic attraction provides a substantially seal between the graft layers 805/807, and the ridges 821 could be felt through the patient's skin, such as in current AVGs, allowing for targeted needle sticks.

In some aspects, the shape and placement of the magnets may be a consideration, for example, some since ring shaped magnets on the ends of the inner lumen may prevent collapse and removal as a small sheath in an endovascular procedure. Instead, whole ring magnets may be placed on the outer graft since it does not need to contract. Regularly-spaced metal tabs or filaments can then be integrated into the inner lumen, enabling magnetic attraction of the two graft layers and narrowing of the inner lumen ends during removal. The device may be produced in bulk length, with the magnet and metal components are spaced apart, allowing the surgeon to cut the graft to the desired length.

Additional aspects to consider during this screening process included difficulty of the insertion and removal process. To keep the insertion method relatively simple and familiar to surgeons, a balloon-catheter-based procedure could be used, but the use of strings and magnets on the graft may affect the alignment of the inner and outer lumen. Furthermore, applying tension to the inner lumen during removal could cause non-uniform changes in its diameter which could complicate the removal process. Other concerns included whether forceps or a hook could be inserted at the incision site during endovascular removal. A secondary device for removal should be small enough to fit through this incision site, yet strong enough to separate the inner lumen from the outer graft. Ease of puncturability was another design aspect to take into account. The two layers should adhere well to each other, but allow for the repetitive puncture that is required for current AVGs; the graft layers should also be able to seal at the sites of the needle sticks to prevent cell build up and bleeding.

Other considerations involved reendothelialization of the inner lumen, which often leads to stenosis in current AVGs. To prevent rapid cell build up and to extend the lifespan of the inner lumen, graft surfaces may be made smooth such that the occurrence of small protrusions in the inner lumen is reduced to minimize cell adhesion.

Additional implementations of a vascular graft with an interchangeable inner lumen include a “finger trap” mechanism, a “drawstring” mechanism, and a double invagination method of removal. For example, sample implementation may include a finger trap PTFE inner lumen with magnetic outer lumen, a pull string PTFE inner lumen with magnetic outer lumen, and a double invagination method of removal for the inner lumen.

A “finger trap” design to facilitate endovascular exchange of the inner lumen of the AVG is shown in FIG. 9. As illustrated in FIG. 9, the “finger trap” design includes an inner lumen 905 made from biaxially braided PTFE fibers 925, that will be helically wound into a cylinder to mimic the traditional finger trap game. Small fragments 927 of iron metal may be embedded within the PTFE fibers 925 around the circumference at one end 909 of the inner lumen 905. A PTFE protrusion on the opposite end may form a small protruding tab 919. The outer lumen may be a tube of PTFE resembling the existing arteriovenous graft, but with a thin neodymium magnet ring 917 embedded within one end of the graft 907. The outer lumen 907 may be surgically inserted into the patient's artery and vein, like the traditional arteriovenous graft. The inner lumen 905 lines the interior of the outer lumen 907 and is held securely in place by the magnetic force between the neodymium ring 917 and the iron fragments 927. By pulling the protruding tab on the inner lumen 905 with forceps, the circumference of the inner lumen 905 contracts, and the length of the inner lumen 905 extends as it is held in tension by the magnetic force. This change in conformation will allow for endovascular removal of the inner lumen 905, as the iron fragments 927 are eventually pulled away from the magnetic rings 917.

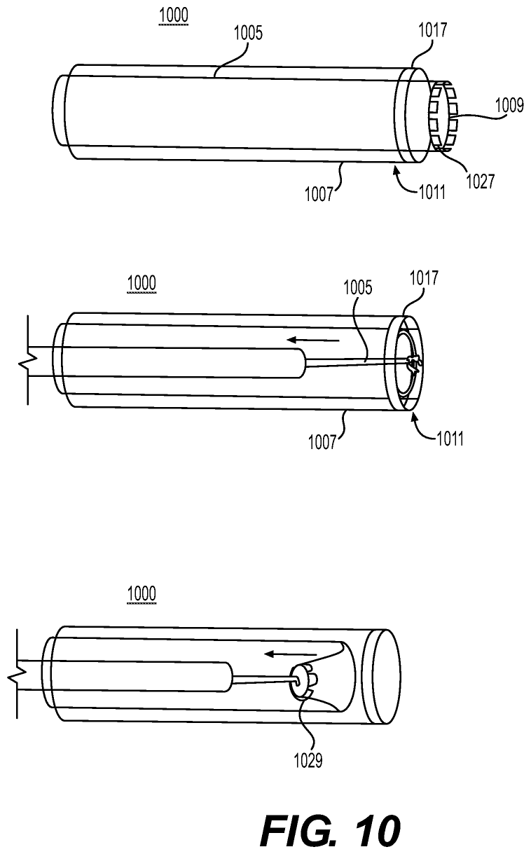

Another embodiment of the device according to principles described herein is illustrated in FIG. 10 and provides a PTFE inner lumen 1005 with iron metal fragments 1027 placed around the circumference of the distal end 1009 that could be removed using a double-step invagination technique. The outer PTFE graft 1007 includes a neodymium magnetic ring 1017 at its distal end 1011; the magnetic attraction between the magnets 1017 and metal regions 1027 attaches the two layers 1005/1007 of the graft 1000. The outer graft 1007 is inserted into the patient's arm using open surgery, while the inner lumen 1005 is inserted using a balloon catheter and a guidewire (not shown), similar to traditional stent placement procedures. To remove the inner lumen 1005, metal Alligator Tooth Retrieval Forceps may be inserted endovascularly to the graft location. Using forceps, the surgeon grabs onto the distal end 1009 of the inner lumen 1005, applying a traction force that causes partial invagination of the inner lumen 1005. A string 1031 could be placed on the distal end 1009 of the inner lumen 1005 to make grasping onto it easier. Continued traction force causes more of the inner lumen 1005 to fold in on itself. To prevent tearing of the inner lumen 1005 while applying traction, a second invagination step may be used. The forceps cause release of the original distal end 1009 of the inner graft 1005 and grasp onto the in-folding edge 1029 of the inner lumen 1005. Applying additional traction force leads to complete removal of the inner lumen 1005 as it is folded inside out. Stretching and in-folding of the inner lumen 1005 decreases its diameter and allow it to be removed endovascularly.

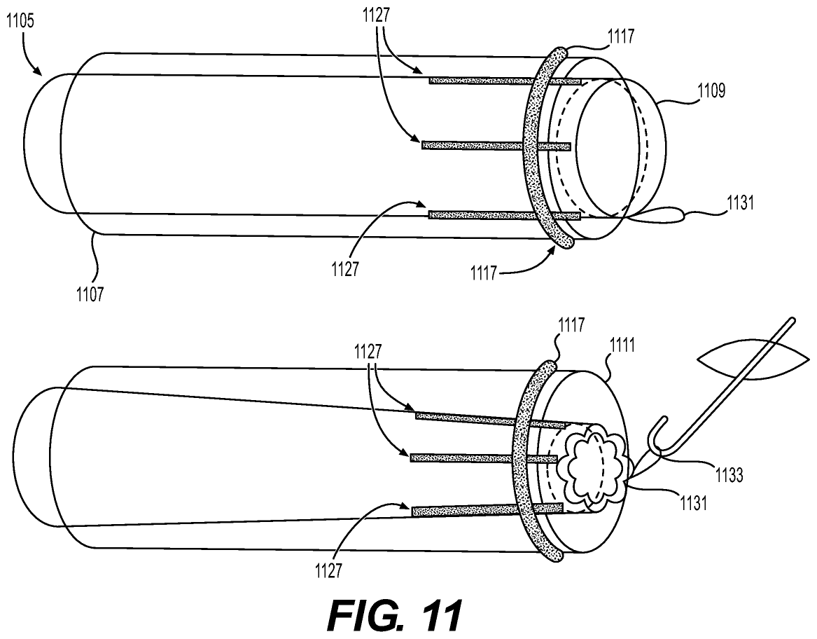

Another embodiment of the device according to principles described herein is illustrated in FIG. 11 and utilizes a “drawstring bag” design to facilitate endovascular exchange of the inner lumen 1105 of the AVG. The second concept utilizes the mechanism of a drawstring bag to facilitate the endovascular exchange of the inner lumen 1105. The design includes an inner lumen made from PTFE with iron filaments 1127 embedded at one end 1109. At the same end 1109 of the inner lumen 1105, a string 1131 is woven through the circumference with a loop 1133 protruding off the end of the graft 1100, modeling a drawstring bag. The outer lumen 1107 may be a tube of PTFE resembling the traditional arteriovenous graft, but with thin, neodymium magnet rings 1117 embedded at the opening of one end 1111.

As in the embodiment illustrated in FIG. 9, the outer lumen will be surgically inserted into the patient's vasculature, and the magnetic force between the neodymium ring 1117 and iron fragments 1127 provides for the inner lumen securely lines the interior of the outer lumen. By pulling the protruding loop 1133 on the inner lumen with forceps or a small hook, the circumference of the inner lumen 1105 will contract, and the iron fragments 1127 pulled away from the neodymium rings 1117, allowing for endovascular removal.

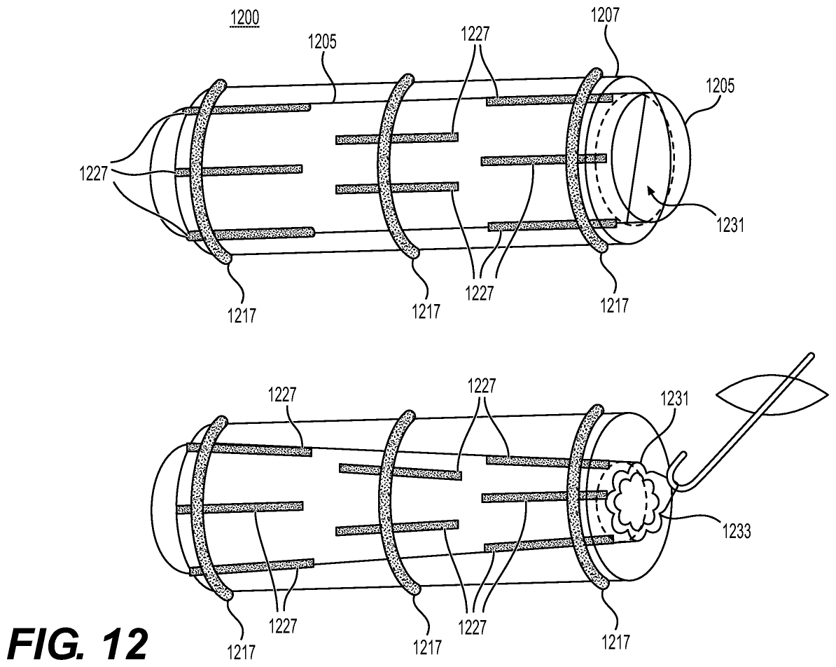

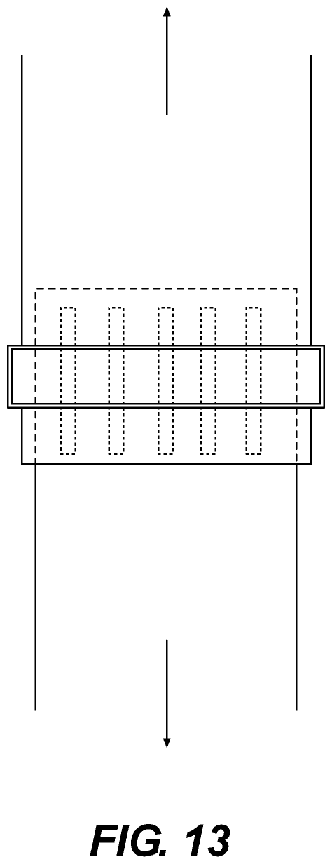

In another aspect of the “drawstring bag” design, the outer graft 1207 may be made of PTFE and have encased neodymium magnets 1217 placed at regular intervals around the whole circumference of the graft (as shown in FIG. 12). These magnets 1217 cause the metal filaments 1227 in the inner lumen 1205 to adhere to the outer lumen 1207. Ridges in the graft at the magnet placement areas can be felt through the skin to indicate areas where a needle cannot be stuck. Because the outer graft 1207 has the magnets 1217 at regular intervals, it can be manufactured in bulk and be cut down to size as physicians require it. The inner lumen 1205 may be a PTFE tube with embedded iron strips 1227 placed at known positions or regular intervals along the tube around the circumference. This is to provide adhesion to the outer graft 1207 at the magnet placement points, as well as the ability to contract radially. At one end of the inner lumen 1205, a polyethylene (courlene) string 1231 may be woven or wrapped in the PTFE with a portion of it traveling through the diameter of the lumen. This alternative placement may provide reduced encapsulation of the string by endothelial cells, which would make endovascular removal very difficult if not impossible. The string 1231 through the diameter will also be in a high flow area, which may further prevent cell adhesion to the string. This component may be used in the removal process to create radial contraction.

The magnetic adhesion method may reduce traveling of the inner lumen 1205 throughout the blood stream and keep the inner lumen 1205 in place during puncturing. Also, the method of adhesion may be relatively easy to implement in an endovascular procedure to keep the implantation and removal procedures from getting too difficult. The pull string 1231 may not require much dexterity to insert or remove.

To insert the inner lumen 1205, a balloon (not shown) may be placed inside the inner lumen 1205, similar to a stent placement procedure. A tube may then be around the balloon and inner lumen to prevent accidental magnetic adhesion during placement. Using x-ray fluoroscopy, the inner lumen can be tracked as it travels through the patient's vasculature. Once the inner lumen is sitting in the proper spot inside the outer graft, the balloon can be inflated and the outer tube can be pulled back to allow radial expansion and adhesion to the magnets on the outer graft. After the inner lumen is fully expanded and adhered, all other tools can be pulled out, and the placement procedure is finished.

To remove the inner lumen, another endovascular procedure using x-ray fluoroscopy may be used. First a small hook can catch the string on the end, and a traction tube can be used to provide some resistance and allow the inner lumen to contract, similar to a drawstring bag. Then a larger tube can go between the inner lumen and outer graft to gently remove them from one another. This minimizes the friction on the outer graft and potentially prolongs its lifespan. Finally, the whole inner lumen can be removed and replaced.

One purpose of the magnets and metal filaments within the device is to hold the inner lumen in place under the shear force of the blood flow. In order to mimic this shear force in test, a prototype of the device may be loaded into a mechanical testing frame (configured as shown in FIG. 13) with the inner and outer portions of the device at opposite ends. Axial force may then be slowly applied. The resulting stress-strain curves collected may be analyzed and the stress values in which the two portions of the device begin to slip will be recorded. This list of stress values may be compared to the shear stress of blood expected within the AVG.

For the inner lumen to function properly for dialysis, it should withstand puncture similar to a typical AVG. When the inner lumen is placed within the outer graft, the graft layers should also adhere firmly together to prevent migration of the device in vivo and to prevent any obstructions to blood flow. The objective of this test is to determine whether the inner lumen allows for puncture without displacing or dislodging the two graft layers significantly.

To begin this test, the fully assembled graft (inner lumen inserted into the outer graft) may be attached to a peristaltic pump with a water flow rate of 1,000 mL/min to simulate blood flow through an AVG; however, a lower flow rate of 500 mL/min may be used depending on the available pump settings. The water flow provides an opposing force to puncture during testing. Once the pump is on, two standard dialysis needles may be inserted through both graft layers in areas that do not have metals or magnets. The regions that are sufficient for puncture are between the magnetic rings, which appear as raised ridges on the outer graft. The tips of the dialysis needles are spaced 6 cm apart as in typical AVGs. For each trial, water may be allowed to flow through the graft for a 10-minute period. During this time, a digital camera may be used to record the initial puncture sites on the outer graft. Because the puncture sites on the inner lumen may be obscured by the outer graft during testing, we assume that the initial puncture diameters for the inner lumen will be the same as the outer graft. The overall diameters of the inner and outer grafts prior to puncture may also be recorded. After flow and puncture testing have been completed, the final diameters of the two graft layers may be recorded. Once the assembled graft has been removed from the peristaltic pump, the inner lumen will be separated from the outer graft, and pictures of the puncture sites on the top portion of the inner lumen will be taken. Each trial may use different puncture sites with the appropriate spacing each time.

To quantify the data, ImageJ software may be used to count the number of cracks that are above 1 mm in length on both the inner and outer graft layers and to measure the diameters of the puncture sites. In addition, ImageJ may be used to measure the diameters of the inner and outer grafts before and after testing. The difference in the graft diameters indicates the amount of separation after flow and puncture tests. To evaluate the results, three-paired samples t-tests may be used. The first paired-samples t-test may compare crack propagation for both the inner and outer grafts. The second t-test may compare the change in the diameter of the puncture sites (difference between initial and final puncture diameters) for both grafts. For this t-test, an average of the change in puncture diameters for the two puncture sites for each trial may be calculated. The third paired-samples t-test may compare the difference in the overall graft diameters before and after puncture and flow testing. If there is no significant difference for any of the comparisons in these t-tests (p-value>0.05), then the durability criteria for the device will have been met.

The clinical benefit of our device lies in the ability of the inner lumen to effectively expand and contract to allow for endovascular replacement. This process provides hemodialysis patients with a way to renew the life of their graft without the need to undergo invasive and costly replacement surgery. The objective of this clinical benefit-based test is to determine the collapsibility of our graft through the removal process and the overall efficacy of the removal process. The collapsibility of the inner lumen will be determined by measuring the change in diameter before and during removal, and the removal efficacy will be determined by determining the percentage of successful removals vs failed removals. Given that there are no specific requirements for incision size of endovascular procedures, any statistically significant change in diameter will be accepted as indicating collapsibility. Removal efficacy will be calculated by the percentage of removals where the inner lumen is fully removed, and the integrity of the remaining outer lumen is preserved.

For further testing, there will be several assumptions made in the interest of devising a simple and effective method of testing. These include the assumption that no blood will be flowing through the lumen at the time of replacement, and that the presence of endothelial cells along the inside of the graft will not have a significant impact on removal of the inner lumen. The procedure will begin with documenting the initial inner lumen diameter within a fully formed graft using a digital camera and physical measurements. The outside of the device will then be affixed to ensure that the outer graft remains in place throughout the removal process. The suture string extending along the face of the inner lumen will be carefully pulled until it is taut using forceps or a small hook to simulate endovascular removal. New measurements of the inner lumen diameter will be taken to the determine the amount of contraction experienced by the inner lumen. The inner lumen will then continue to be carefully removed by pulling the suture string until it is completely removed from the outer graft or until damage occurs. To determine if the removal of the inner lumen was a success, observations will be continually recorded throughout the testing, to ensure that the inner lumen is completely removed, and that no damage occurs to the outer lumen. This process will be repeated 10 times to gain an adequate amount of data for analysis. ImageJ software will be utilized to take precise measurements of the inner lumen diameters from the digital camera images collected and determine the amount of contraction for each trial. A paired t test will be used to determine if the contraction of the inner lumen is statistically significant, and the percentage of successful removals will be calculated to calculate the device's clinical benefit.

As discussed herein, the present designs are intended to provide physicians with an inner lumen for the AVG that can be replaced endovascularly so as to decrease the frequency of intervention required for patients undergoing hemodialysis treatments. Features of the AVG include application for patients not suited for AVF, connecting an artery to a vein, act as an access point for hemodialysis treatment, prone to stenosis, thrombosis and pseudoaneurysms, require frequently intervention via, for example, balloon angioplasty, made typically of PTFE, low biodegradability and high endothelization. In any of the embodiments described herein, the inner lumen may comprise PCL in HFP, may include nanoparticles in some designs described herein and may be placed within a standard PTFE outer graft/lumen.

In analysis of some of the various embodiments as described herein, testing was performed by attaching an example graft to a peristaltic pump. The pump was set to a flow rate of 500 mL/min. Prior to testing a length, proximal diameter and distal diameters of the graft were measured. The pump was then activated and water was pumped through the graft for 14 minutes. Following termination of the pump flow, the proximal and distal diameters were recorded. Any water leakage and damage to the graft was recorded. In clinical analysis of some of the various embodiments described herein, a graft according to principles described herein was attached to a peristaltic pump. A dialysis needle was then used to puncture one layer of a middle section of the graft. With the dialysis needle inserted into the graft, water was pumped through the graft at a rate of 500 mL/min for 14 minutes. Any water leakage and damage to the graft was recorded.

While hemodialysis is an effective means of treating end stage kidney disease, the frequent access to the patient's vasculature, and the high rate of blood flow required for the dialysis machine present complications. Patients who treat their end-stage kidney disease with hemodialysis typically must undergo the treatment three times a week The arteriovenous fistula (AVF) is the most favored option for vascular access. This method has resulted in the most reliable and efficient hemodialysis performance and can be used for many years making it a good long-term method of vascular access. However, the creation of an AVF requires that the patient has suitable veins to form a fistula, and the maturation period for the fistula can be up to twelve weeks.

The central venous catheter (CVC) is a strong temporary option for gaining quick and easy vascular access, but poses a high danger of infection, complications, and injury. As a result, patients with a CVC experience many limitations and must take excessive precautions when using one, making this option commonly only used for a shorter time span of weeks to months.

The arteriovenous graft (AVG) is an alternative option for patients who are not suitable for the AVF or the CVC. The AVG creates the high blood flow rate required to the dialysis machine by feeding the high volume of blood within the patient's artery through their smaller vein, and has a maturation period of only three to four weeks, with a lifespan of up to two to three years The AVG is most commonly made from polytetrafluoroethylene (PTFE). PTFE is a carbon and fluorine-based hydrophobic polymer that is non-biologically degradable as well as biologically inert within the human body. A standard AVG made from PTFE is initially around 70 cm long and then cut by the surgeon prior to the graft placement in order to create the appropriate length for the patient. The graft is then inserted surgically using local anesthesia and is typically placed in the patient's forearm in either a straight or looped orientation.

While the AVG solves some issues posed by alternative methods, there are many risks and complications preventing it from being a more widely used option. Stenosis is the abnormal narrowing of a blood vessel, and the most common cause of AVG dysfunction. In an AVG, stenosis is typically triggered by damage to endothelial cells resulting in upregulation of adhesion molecules and migration and proliferation of additional cell types. Thrombosis is the formation of a blood clot within a vessel which results in the obstruction of blood flow. In an AVG, thrombosis is primarily caused by stenosis. A pseudoaneurysm occurs when a blood vessel is damaged causing the leakage of blood which is then contained by the surrounding tissue. In an AVG pseudoaneurysms are rare but may occur in spots that have experienced multiple needle punctures, and can cause thrombosis.

The primary method of treating AVG complications like stenosis and preventing further complications like thrombosis is balloon angioplasty. Balloon angioplasty has a high immediate success rate and is performed percutaneously by inserting a catheter with a deflated balloon into the affected portion of the blood vessel and inflating the balloon to expand the area of stenosis. Stent placement and surgical revision are alternative means of intervention but are generally not recommended except for special circumstances in order to preserve the patient's native vasculature. The various complications associated with the demands of hemodialysis and each method of vascular access indicate the need for a safer and more effective solution. Since there have not been significant advancements related to AVG revision in the past year, our team aims to create a device that functions as an AVG, but solves the complications associated with the AVG in a less invasive, more effective, and more permanent manner than current methods.

This device intends to fulfill the need for a method of vascular access that has similar advantages to that of the AVG but results in complications that are fewer in number and magnitude. The AVG is the ideal option for a patient who needs a long term and readily available means of vascular access for hemodialysis and does not have suitable vein options for the creation of a fistula. AVF nonmaturation is also a growing problem, which is contributing to the switch to AVG for many hemodialysis patients. Approximately 15-20% of hemodialysis patients rely on AVG after only their first year. Currently, the complications associated with the traditional AVG result in the need for the patient to undergo frequent interventions and graft revisions every three to four months after only the first year of AVG use.

The device may allow hemodialysis patients who rely on an AVG to gain the vascular access required for hemodialysis and experience less complications and interventions while doing so.

The scope of our device design is meant to include patients with chronic renal kidney failure that rely on hemodialysis, and who also utilize the AVG method of vascular access to their upper extremities. Patients with an AVG typically undergo hemodialysis several times a week and need a long-term option for vascular access. These patients typically also do not have arteries and veins that are adequate size and depth to support the formation of an AVF and may also have had issues with the nonmaturation of AVF in the past. The scope of this design could also include hemodialysis patients with end-stage kidney disease who currently use a central venous catheter or AVF, but could benefit from the creation of a more safe and reliable form of AVG. While an AVG can be placed across the chest or in lower extremities, the instances of this are extremely rare, and each location presents its own risks and unique challenges for placement, which will not be accounted for in the scope of this design. This will allow for better focus on addressing the specific design requirements of the upper extremity AVG, and to provide better developed options and research for the largest subset of hemodialysis patients. The intended environments for use of the device will be a clinical setting in which a surgeon or other trained medical professional can gain endovascular access and replace the graft's inner lumen, or in an outpatient setting like a hemodialysis center or the patient's home where hemodialysis will be performed by either a nurse/technician or the patient.

The creation of a graft with an interchangeable lumen has potential applications beyond dialysis, especially with regard to covered stents or any bypass graft often used in the treatment of coronary artery disease (CAD) and other cardiovascular problems. Covered stents are metal stents surrounded by a PTFE membrane designed to prevent restenosis due to tissue build up through the stent mesh. Common complications associated with covered stent implantation include fracture, in-stent restenosis, infolding of the stent, and the formation of aneurysms. Also, there have been cases of lumen loss at the edges of these types of stents depending on if the PTFE layer covers the ends of the stent. Coronary artery bypass graft surgery involves taking a vein or artery from another region of the body; one end of this graft is attached above the blocked portion of the coronary artery while the other end is attached below this blocked portion, allowing blood to bypass the blockage and reach the heart. Plaque formation and later plaque rupture can lead to thrombosis and the obstruction of blood flow, leading to the failure of the graft in some patients. These complications such as stenosis and thrombosis are similar to issues encountered when using an AVG. By using an exchangeable lumen within a covered stent or within a bypass graft, the need for invasive surgery will be minimized and the frequency of surgical intervention to correct blockages and narrowing of the graft will be reduced.

While the existing AVG meets many of the needs of patients with end-stage renal failure who rely on long term and frequent hemodialysis, the complications and limitations associated with the AVG highlight the need for a new assistive device. The current standard AVG is a simple tube made from PTFE which commonly experiences stenosis and damage given the high rate of blood flow it supports, and the frequent needle sticks required for transfer of blood into the dialyzer. The new device design will be a vascular graft with an inner lumen that can be replaced endovascularly, resulting in a clean graft without the requirement of invasive surgeries.

This device design may involve an initial surgical procedure in which the device is placed, and then upon the first need of intervention, the complete removal and replacement of the graft's inner lumen may be performed endovascularly. In the design of the device, subsequent interventions may be delayed given the completely renewed interior lumen of the graft and fully renewed lifetime of the graft.

Expanded PTFE (ePTFE) may be used in any of the AVG assemblies described herein in place of PTFE, for example to make any tube described herein. Expanded PTFE results in a microporous tube that allows for air, making the material soft and flexible. In an aspect of an AVG according to principles described herein, the device may include biocompatible polymer for both the inner and outer layers, as well as ePTFE for the inner layer (AV grafts with rapid post-operative self-sealing capabilities). This allows for self-sealing because using ePTFE, like PTFE, is not typically self-sealing after being punctured (AV grafts with rapid post-operative self-sealing capabilities). Previous self-sealing grafts have not been as successful as this specific device due to their bulkiness and lack of tissue growth to heal (AV grafts with rapid post-operative self-sealing capabilities). The other patent that uses ePTFE is a stented vascular graft. This vascular graft uses ePTFE for the stent allowing for it to self-expand as an alternative to the typical balloon expanding stents that are used. The graft may comprise multiple layers of biocompatible polymer for the outer layer and/or the outer layer.

Embodiments of an AVG with exchangeable inner lumen according to principles described herein seek to provide various advantages, although not all embodiments necessarily satisfy all advantages. These advantages include:
a device that can act as a fully functional AVG, while also reducing the occurrence of common complications by allowing for endovascular replacement;
biocompatibility, which will allow for the promotion of endothelial tissue which is enhances functionality of the graft;
graft function and safety, e.g., mechanical properties to remain effective when subjected to the forces of blood flow in the cardiovascular system, which may prevent damage to the graft when it is in use and reduce resulting complications such as stenosis;
non-biodegradability, which ensures that the device does not degrade in the body over time and preserves the integrity of the other graft properties over the lifespan of the graft;
graft supply to the dialyzer with vascular access, allowing patients that undergo hemodialysis at home or in outpatient settings;
for the inner lumen to be endovascularly replaced through a small incision, which would make the process of replacing the graft much less invasive; and
for the device to have a long lifespan, which reduces the interventions experienced by the patient and increases the reliability of the device.

Hemodialysis is a treatment that many people with end stage kidney disease rely on several times a week to filter and balance solutes within the blood. Each treatment of hemodialysis requires frequent and reliable access to the patient's vasculature. The current optimal method of vascular access is AVF, which is associated with high efficacy and low risk of complication. However, many patients who rely on hemodialysis do not have suitable veins for the creation of a fistula. or may not have the vascularization to allow for maturation of the arteriovenous fistula. AVG is the alternative option of long-term vascular access for hemodialysis for these patients. However, the AVG is associated with several complications which can occur frequently after the first year of use. These complications require graft revision which sacrifice the functionality of the graft, and become required regularly. The frequency and consequences associated with the AVG created the motivation to produce this new device. The new device will act as a fully functional AVG but will include an inner lumen which is able to be endovascularly replaced once complications are detected, renewing the lifespan and functionality of the graft.

One of the primary users of this device will be hemodialysis patients that have insufficient vasculature for an AV fistula, especially elderly patients. The proportion of people who are 75 and older when starting dialysis is increasing; 75% of them have five or comorbidities and over 90% of these patients have cardiovascular disease which increases the likelihood that the fistula will fail to mature. Old age in addition to underlying medical conditions contribute to the failure of AVFs; therefore, they are not recommended for elderly patients with low life expectancy. The AVG graft becomes a much more viable option for vascular access for older patients and is often used as the second line of treatment following the failure of an AVF. As of 2018, 20.6% of patients receiving hemodialysis that were 75 and older used an AV graft compared with 13.1% of patients aged 18-44 years. Common complications of the AVG such as stenosis and thrombosis require frequent surgical intervention to restore the function of the graft while other complications such as infection may call for total or partial removal of the graft. Endovascular removal of the inner lumen limits the need for open surgery, reduces complications, and quickens recovery time, leading to shorter hospital stays. Compared to an open surgery, an endovascular procedure is much less invasive and usually only requires a needle puncture and a sheath (Tucson Osteopathic Medical Foundation News, n.d.). With this new device, open surgery may only be needed for initial graft placement while the replacement of the inner lumen may be performed endovascularly, likely as an outpatient procedure.

In addition, the lifespan of the inner lumen of at least 6 months or more reduces the frequency of surgical intervention, reducing medical costs for patients and the time spent obtaining secondary procedures. Also, patients with coronary heart disease could potentially use this device as the removable inner lumen concept can be applied to covered stents or any bypass graft. Covered stents require large vascular access and can cause complications such as stent fracture, restenosis, and aneurysm formation. Coronary artery bypass grafts require invasive surgery and may need coronary angioplasty to widen the arteries as a secondary procedure. Replacement of the inner lumen for a covered stent or bypass graft would minimize the invasiveness of these procedures, reduce complications, and reduce the frequency of surgical intervention. Physicians, especially vascular surgeons, are stakeholders in this device as well. Because the inner lumen can be removed and replaced endovascularly, there is lower surgical risk compared to open surgery, and surgeons will be more likely to perform a procedure involving this device.

Medical insurance companies will also benefit from a graft with an interchangeable lumen since the graft will not need frequent replacement thereby lowering costs. Typically, insurance companies cover the cost for dialysis as well as the implementation and maintenance of the vascular access method. The average monthly cost of an AVG for hemodialysis including treatments for resulting complications is $9,605. The minimal risk involved in the insertion and replacement of the inner lumen and reduced surgical intervention required to address stenosis and thrombosis will help decrease monthly costs for insurance companies and out-of-pocket costs for patients. Hospitals can also benefit as the graft with an inner lumen could be available as a bulk buy, lowering the overall cost of the device. Premade graft tubes with the inner lumen already adhered to the outer graft could be manufactured; the surgeon can then cut the tube to the desired length similar to existing AVGs.

The expected lifespan of the disclosed device is expected to be 6-8 months before it will need to be replaced. The outer lumen will stay within the patient while the inner lumen will be replaced every 6 to 8 months. After the removal of the inner lumen, it will be disposed of following the Environmental Protection Agency's (EPA) medical waste standard in Ohio. In Ohio, most medical wastes are incinerated or autoclaved (How Waste Incineration Works-Earth911. (n.d.)). However, the gases released into the environment from the inner lumen may be toxic. Alternative options that could be used to dispose of the waste are thermal treatments, chemical treatments, and electropyrolysis.

As described herein, an arteriovenous graft device may include an outer conduit having a first outer conduit end and a second outer conduit end opposite the first outer conduit end, the first outer conduit end defining an outer conduit opening extending from the first outer conduit end to the second outer conduit end, wherein the first outer conduit end is configured to be in fluid communication with an artery and the second outer conduit end is configured to be in fluid communication with a vein; and an inner conduit defining an inner conduit opening, wherein the inner conduit is sized to be disposed within the outer conduit opening, wherein the inner conduit is movable between a collapsed position and an expandable position.

In an aspect of the device, the inner conduit and the outer conduit may comprise the same material. In an aspect of the device, the inner conduit the inner conduit comprises nitinol. In an aspect of the device, the inner conduit one of the inner conduit and the outer conduit comprises magnetic materials. In an aspect of the device, the inner conduit the magnetic materials comprise neodymium, Fe₃O₄ or FePt. In an aspect of the device, the inner conduit another of the inner conduit and the outer conduit comprises metal materials. In an aspect of the device, the inner conduit at least one of the inner conduit and the outer conduit comprises polytetrafluoroethylene (PTFE). In an aspect of the device, the inner conduit the inner conduit comprises 10% polycaprolactone (PCL) in hexafluoropropane (HFP). In an aspect of the device, the inner conduit the inner conduit comprises a hydrophobic material. In an aspect of the device, the inner conduit the inner conduit comprises a biocompatible material.

As described herein a method of inserting an inner conduit into an outer conduit of an arteriovenous graft device may include providing an outer conduit having a first outer conduit end and a second outer conduit end opposite the first outer conduit end, the first outer conduit end defining an outer conduit opening extending from the first outer conduit end to the second outer conduit end, wherein the first outer conduit end is in fluid communication with an artery and the second outer conduit end is in fluid communication with a vein; advancing an inner conduit coupled to a guidewire through a portion of the artery to the first outer conduit end, the inner conduit defining an inner conduit opening, wherein the inner conduit is sized to be disposed within the outer conduit opening, wherein the inner conduit is in a collapsed position and is movable between a collapsed position and an expandable position; inserting the inner conduit into the outer conduit opening; and moving the inner conduit from the collapsed position to the expanded position.

In an aspect of the method, the inner conduit and the outer conduit comprise the same material. In an aspect of the method, the inner conduit comprises nitinol. In an aspect of the method, one of the inner conduit and the outer conduit comprises magnetic materials. In an aspect of the method, the magnetic materials comprise, neodymium, Fe₃O₄ or FePt. In an aspect of the method, another of the inner conduit and the outer conduit comprises metal materials. In an aspect of the method, at least one of the inner conduit and the outer conduit comprises polytetrafluoroethylene (PTFE). In an aspect of the method, the inner conduit comprises 10% polycaprolactone (PCL) in hexafluoropropane (HFP). In an aspect of the method, the inner conduit comprises a hydrophobic material. In an aspect of the method, the inner conduit comprises a biocompatible material.

As described herein, a method of removing an inner conduit from an outer conduit of an arteriovenous graft device may include providing an arteriovenous graft device, the device having an outer conduit having a first outer conduit end and a second outer conduit end opposite the first outer conduit end, the first outer conduit end defining an outer conduit opening extending from the first outer conduit end to the second outer conduit end, wherein the first outer conduit end is in fluid communication with an artery and the second outer conduit end is in fluid communication with a vein, and an inner conduit defining an inner conduit opening, wherein the inner conduit is disposed within the outer conduit opening, wherein the inner conduit is in an expanded position and is movable between a collapsed position and an expandable position; advancing a guidewire through a portion of the artery to the inner conduit; coupling the guidewire to the inner conduit; moving the inner conduit from the expanded position to the collapsed position; removing the inner conduit from the outer conduit opening; and removing the guidewire and inner conduit from the artery.

In an aspect of the method, the inner conduit and the outer conduit comprise the same material. In an aspect of the method, the inner conduit comprises nitinol. In an aspect of the method, one of the inner conduit and the outer conduit comprises magnetic materials. In an aspect of the method, the magnetic nanoparticles comprise neodymium, Fe₃O₄ or FePt. In an aspect of the method, another of the inner conduit and the outer conduit comprises metal materials. In an aspect of the method, at least one of the inner conduit and the outer conduit comprises polytetrafluoroethylene (PTFE). In an aspect of the method, the inner conduit comprises 10% polycaprolactone (PCL) in hexafluoropropane (HFP). In an aspect of the method, the inner conduit comprises a hydrophobic material. In an aspect of the method, the inner conduit comprises a biocompatible material.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Various implementations include an arteriovenous graft device. The device includes an outer conduit and an inner conduit. The outer conduit has a first outer conduit end and a second outer conduit end opposite the first outer conduit end. The first outer conduit end defines an outer conduit opening extending from the first outer conduit end to the second outer conduit end. The first outer conduit end is configured to be in fluid communication with an artery and the second outer conduit end is configured to be in fluid communication with a vein. The inner conduit defines an inner conduit opening. The inner conduit is sized to be disposed within the outer conduit opening. The inner conduit is movable between a collapsed position and an expandable position.

Various other implementations include a method of inserting an inner conduit into an outer conduit of an arteriovenous graft device. The method includes providing an outer conduit, such as the outer conduit described above, and placing the first outer conduit end in fluid communication with an artery and the second outer conduit end in fluid communication with a vein. The method further includes advancing an inner conduit, such as the inner conduit described above, coupled to a guidewire through a portion of the artery to the first outer conduit end. The inner conduit is in a collapsed position and is movable between a collapsed position and an expandable position. The method further includes inserting the inner conduit into the outer conduit opening and moving the inner conduit from the collapsed position to the expanded position.

Various other implementations include a method of removing an inner conduit from an outer conduit of an arteriovenous graft device. The method includes providing an arteriovenous graft device, such as the device described above; advancing a guidewire through a portion of the artery to the inner conduit; coupling the guidewire to the inner conduit; moving the inner conduit from the expanded position to the collapsed position; removing the inner conduit from the outer conduit opening; and removing the guidewire and inner conduit from the artery.

While various embodiments of the present invention have been described above, it should be understood that they have been presented by way of example only, and not limitation. It will be apparent to persons skilled in the relevant art that various changes in form and detail can be made therein without departing from the spirit and scope of the present invention. Thus, the breadth and scope of the present invention should not be limited by any of the above-described exemplary embodiments, but should be defined only in accordance with the following claims and their equivalents.

What is claimed is:

1. An arteriovenous graft device, the device comprising:
an outer conduit having a first outer conduit end and a second outer conduit end opposite the first outer conduit end, the first outer conduit end defining an outer conduit opening extending from the first outer conduit end to the second outer conduit end, wherein the first outer conduit end is configured to be in fluid communication with an artery and the second outer conduit end is configured to be in fluid communication with a vein; and
an inner conduit defining an inner conduit opening, wherein the inner conduit is sized to be disposed within the outer conduit opening, wherein the inner conduit is movable between a collapsed position and an expandable position.

2. The device of claim 1, wherein the inner conduit and the outer conduit comprise the same material.

3. The device of claim 1, wherein the inner conduit comprises nitinol.

4. The device of claim 1, wherein one of the inner conduit and the outer conduit comprises magnetic materials.

5. The device of claim 4, wherein the magnetic materials comprise neodymium, Fe₃O₄ or FePt.

6. The device of claim 4, wherein another of the inner conduit and the outer conduit comprises metal materials.

7. The device of claim 1, wherein at least one of the inner conduit and the outer conduit comprises polytetrafluoroethylene (PTFE).

8. The device of claim 1, wherein the inner conduit comprises 10% polycaprolactone (PCL) in hexafluoropropane (HFP).

9. The device of claim 1, wherein the inner conduit comprises a hydrophobic material.

10. The device of claim 1, wherein the inner conduit comprises a biocompatible material.

11. A method of inserting an inner conduit into an outer conduit of an arteriovenous graft device, the method comprising:
providing an outer conduit having a first outer conduit end and a second outer conduit end opposite the first outer conduit end, the first outer conduit end defining an outer conduit opening extending from the first outer conduit end to the second outer conduit end, wherein the first outer conduit end is in fluid communication with an artery and the second outer conduit end is in fluid communication with a vein;
advancing an inner conduit coupled to a guidewire through a portion of the artery to the first outer conduit end, the inner conduit defining an inner conduit opening, wherein the inner conduit is sized to be disposed within the outer conduit opening, wherein the inner conduit is in a collapsed position and is movable between a collapsed position and an expandable position;
inserting the inner conduit into the outer conduit opening; and
moving the inner conduit from the collapsed position to the expanded position.

12. The method of claim 11, wherein the inner conduit and the outer conduit comprise the same material.

13. The method of claim 11, wherein the inner conduit comprises nitinol.

14. The method of claim 11, wherein one of the inner conduit and the outer conduit comprises magnetic materials.

15. The method of claim 14, wherein the magnetic materials comprise, neodymium, Fe₃O₄ or FePt.

16. The method of claim 14, wherein another of the inner conduit and the outer conduit comprises metal materials.

17. The method of claim 11, wherein at least one of the inner conduit and the outer conduit comprises polytetrafluoroethylene (PTFE).

18. The method of claim 11, wherein the inner conduit comprises 10% polycaprolactone (PCL) in hexafluoropropane (HFP).

19. The method of claim 11, wherein the inner conduit comprises a hydrophobic material.

20. The method of claim 11, wherein the inner conduit comprises a biocompatible material.
